(19) United States
(12) Patent Application Publication
(10) Pub. No.: US 2022/0122715 A1
(43) Pub. Date: Apr. 21, 2022

(54) METHOD AND SYSTEM FOR GENERATING A THERAPEUTIC SESSION

(71) Applicant: ONCOMFORT SA, Wavre (BE)

(72) Inventors: Diane Jooris, Tervuren (BE); Julien Tesse, Bruxelles (BE)

(73) Assignee: ONCOMFORT SA, Wavre (BE)

(21) Appl. No.: 17/425,772

(22) PCT Filed: Jan. 28, 2020
(86) PCT No.: PCT/EP2020/052025

§ 371 Date: Jul. 26, 2021

(30) Foreign Application Priority Data
Jan. 28, 2019 (BE) 2019/5046

(51) Int. Cl. G16H 20/70; G06F 3/01; G06F 3/048; A61M 21/02

(52) U.S. Cl. CPC G16H 20/70; G06F 3/016; A61M 2021/005; A61M 21/02; G06F 3/048

(57) ABSTRACT

The present invention is in the field of clinical therapy. In particular, the present invention provides systems and methods for generating at least a part of a therapeutic session for controlling a subject via sensory stimulations, such as graphical and/or auditory stimulations.

### TECHNICAL FIELD

The present invention is in the field of clinical therapy. In particular, the present invention provides systems and methods for generating at least a part of a therapeutic session for controlling a subject via sensory stimulations, such as graphical and/or auditory stimulations.

### BACKGROUND

Therapeutic sessions can be used in a clinical environment for inducing an altered state of consciousness (ASC) in a subject. While immersed in the altered state of consciousness, the subject self-perception and the peripheral awareness are affected, changing the subject's experience of his/her sensation, perception, and thoughts, and making the subject prone to suggestive control of his/her physiological responses and movements.

There exist various techniques that may aid in altering a subject's state of consciousness. One method observed to be particularly effective involves inducing a hypnotic state in a subject. The hypnotic state has been described as a state of consciousness with an associated heightened level of suggestibility. While in this state the experience of sensations, perceptions, thoughts, or behaviour may change significantly. The hypnotic state may be induced using drugs, concentration (i.e. focusing attention on a specific object) and sensory information such as imagery/audio; of the listed categories imagery/audio is believed to be the most promising as it can produce the desired state of consciousness (with the associated heightened level of suggestibility) without the stress of concentration (which also produces an unrelaxed state) or the side effects of drug-induced states. Various other psychological techniques can also contribute to the altered state of consciousness.

In a clinical environment the hypnotic state will typically be induced using clinical hypnosis therapy delivered by trained health professionals. This therapy may be performed by exposing a subject to a series of auditory stimulations and graphical/imagery suggestions, which help relax the subject and place him/her in an immersed state characterised by reduction or absence of movement, and dissociation of reality. Once fully immersed, the subject's self-perception and peripheral awareness are modified to the extent that his/her physical responses become controllable; for example, the hypnotic state may allow for management of anxiety and pain, or for influencing the respiratory motions such as the respiratory rate and inhalation-to-exhalation ratio. In recent years more and more studies have been performed to recognize the possibilities of such therapy during medical procedures. For instance, in one study (Liu Y. et al.; Radiotherapy and Oncology 2018; 127: 390) clinical hypnosis was applied to better control the respiratory motions of subjects for radiotherapy of lung cancer, which allowed easier probe placement and medical imaging.

However, performing such therapies in a clinical environment is often a challenging task requiring the presence of specialised personnel. Achieving sufficient control over the subject's senses and motions can be difficult without proper guidance. These are cumbersome tasks for medical practitioners, who are oftentimes too preoccupied with other medical tasks to give attention to the subject's state of consciousness. US 2010/010289 describes an exemplary medical hypnosis device for controlling the administration of a hypnosis experience through audio or visual means. The device may comprise a number of sensors for measuring physiological parameters of the user. Data from the sensors is used for determining the neurological state of a subject, such that instructions or suggestions directed at the user's subconscious mind are delivered at an appropriate time during the hypnosis experience.

When used during a medical procedure, clinical hypnosis suggestions may be adapted to the environment and sequences of the medical procedures. Metaphors, instructions and suggestions can be added to include specific events of the medical intervention into the dissociative experience. However, despite the provision of a warning message to the subject, any action performed by a medical practitioner near the subject may disrupt the altered state of consciousness and leave the subject in a stressed state. For example, the practitioner may need to grab or touch the subject's body, disinfect the skin with a (cold-feeling) disinfectant, use of loud machinery, use of chemicals with unpleasant smells, treating burns, and so on. Uncontrolled interruption of the altered state of consciousness may not only negate the positive effects of the therapeutic session, but may also have adverse effects on the subjects, such as increasing the level of anxiety or letting the subject feel confused. Also pre-existing feelings of pain, anxiety or any type of emotional or physical discomfort may prevent the subject from becoming fully immersed in the session. Present systems fail to address these issues.

Accordingly, there is a need for systems, devices and/or methods that can aid in achieving an altered state of consciousness in subjects during a therapeutic session, such as in a clinical environment. Preferably, the system may aid in controlling the subject's perceptions and responses during the therapeutic session. Preferably, the system may aid in preventing the altered state of consciousness from being disrupted by an external action or stimulus. Preferably, the system may also ensure that a (medical) procedure can be performed smoothly with improved collaboration from a subject.

In the recent past, there has been a growth of research efforts involving heart rate variability. By definition, heart rate variability (HRV) is the variation of the time interval between two succeeding heartbeats (in ms). The HRV is not only considered a useful indicator of health-related issues, but it has also been observed to be directly linked with the autonomic nervous system. For instance, the parasympathetic nervous system, also known as the ‘rest-and-digest’ system, can use the vagus nerve to lower heart rate and increase HRV.

It is widely accepted that multitude of respiratory parameters may affect the HRV (Tripathi; Ind J Aerospace Med 2004; 48(1): 64-75); these parameters may include, amongst others, the respiratory rate (Schipke J D, et al.; J Clin Basic Cardiol 1999; 2: 92-4) and the relative timing of expiration/inspiration (Strauss-Blasche G et al.; Clin Exp Pharmacol Physiol 2000; 27: 601-6). Indeed, slow-paced breathing techniques were observed to increase the HRV of a subject while also providing a feeling of relaxation (Kromenacker B W et al.; Psychosom Med. 2018; 80(6):581-587). Other research confirmed that the inhalation-to-exhalation ratio is an important modulator of the autonomic nervous system (Van Diest I et al; Appl Psychophysiol Biofeedback 2014; 29(3-4):171-80). In other words: not only can breathing techniques exert control over the HRV, they may also stimulate the parasympathetic nervous system. Accordingly, there is also need for a system that may aid in controlling the subject's body response during a therapeutic session, such as the heart rate and respiratory parameters.

### SUMMARY

Described herein is a computer-implemented method for generating at least a part of a therapeutic session, the method comprising:
providing a first database comprising a plurality of blocks, each representing a sensory information, preferably a visual and/or audio segment, to be executed in its entirety,
generating a script defining at least a part of the therapeutic session, the script instructing sequential execution of at least 2 entire blocks selected from the first database;
storing the script for subsequent execution of the blocks whereby the visual and/or audio segments are presented to a subject during the therapeutic session.

The first database may further comprise one or more event class blocks comprising an asset that is sensory information such as a visual and/or audio element configured to guide the perception of the subject; wherein the method further comprises:
receiving a user input during execution of the script of the therapeutic session of a requested event;
selecting from the first database at least 1 event block responsive to the user input;
inserting into the stored script an instruction for execution of the selected event block without interruption of the execution of the current block.

The inserting of the at least 1 event class block into the stored session may comprise:
inserting the selected event block between two existing blocks in the script to be executed after the current block; and/or
merging the selected event block with one or more existing blocks in the script to be executed during or after the current block.

The method may further comprise:
providing an output for a GUI indicating the insertion of the selected event block into the progress of the session, and optionally providing a visual indicator indicating the remaining time until execution of the selected event, preferably via a countdown timer.

The first database may further comprise one or more outcome class blocks comprising an asset that is sensory information such as a visual and/or audio element of the segment configured to achieve a specific effect on the subject; wherein the method further comprises:
receiving a user input prior to generation of the script of a requested outcome of the session;
   wherein the step of generating comprises selecting from the first database at least 1 outcome class block comprising an asset that is sensory information such as a visual and/or audio element of the segment configured to achieve a specific effect on the subject corresponding to the requested outcome of the session;
   and/or, wherein the method further comprises:
receiving a user input during execution of the script of a requested outcome of the session;
selecting from the first database at least 1 outcome block responsive to the user input;
inserting into the stored script an instruction for execution of the selected outcome block without interruption of the execution of the current block.

The inserting of the at least 1 outcome class block into the stored session may comprise:
inserting the selected outcome block between two existing blocks in the script to be executed after the current block; and/or
merging the selected outcome block with one or more existing blocks in the script to be executed during or after the current block.

The method may further comprise:
receiving a user input prior to generation of the script of a requested total duration of the session;
   wherein the step of selecting comprises selecting blocks from the first database such that a sum of the selected block durations corresponds with the requested total duration of the stored session.

The method may further comprise:
receiving a user input during execution of a current block of an adjusted total duration of the session;
selecting one or more blocks for adding to or removing from the stored script without interruption of the execution of the current block such that a sum of the selected block durations extends or reduces the duration of the session according to the requested adjusted total duration of the stored session.

The method may further comprise:

providing an output for a graphical user interface (GUI) indicating progress of the session and total duration of the session.

At least one of the blocks in the first database may be classified as a breathing class block comprising an asset that is a visual and/or audio element of the segment configured to induce a defined respiratory pattern in the subject, preferably a defined respiratory rate and/or the inhalation-to-exhalation ratio.

The method may select at least two breathing class blocks to be played sequentially; wherein a first breathing class block has a respiratory pattern corresponding with a standard respiratory pattern of the subject, the second breathing class block has a respiratory pattern corresponding with an adjusted respiratory pattern.

The method may select a sequence of at least two breathing class blocks to be played sequentially; wherein each breathing class block in the sequence defines a constant respiratory pattern and is evolved with respect to an adjacent breathing class block.

The evolution may be an incremental change optionally that is 10 to 50%; more preferably 20 to 40%; most preferably 25 to 35%; for example 33% of the inhalation and/or exhalation duration.

The asset of the breathing class block may be at least one visual or audio asset, the asset or part thereof having a definable motion rate, DMR, to which the respiratory pattern of the subject synchronises, wherein the DMR is defined by a change in position of the graphical element between a first region and a second region within a set time interval.

The asset of the breathing class block may be at least one audio asset repeating at a set time interval to which the respiratory pattern of the subject synchronises.

The method may comprise providing an output for a GUI indicating the respiratory pattern and progress of the current breathing block, and optionally the respiratory patterns and running times of a following block.

The therapeutic session may comprise at least two phases of: (i) an induction phase, (ii) a deepening phase, (iii) a transition phase and (iv) a re-alerting phase, wherein each phase comprising at least partially different blocks; and wherein a script defining at least the deepening phase, and one other phase is generated.

Each block may be assigned a unique identity code in the first database, and a second database contains a list of unique identity codes of the first database, each unique identity code associated with a block reference parameter providing reference information about the block, wherein the selecting comprises consultation of the second database to determine the blocks selected from the first database to generate the therapeutic session.

A block reference parameter associated with the unique identity code is one or more of the following:
a class indicator, indicating the class of the block, where a block class is indicative of an effect on a subject or lack thereof;
a phase identifier, indicating which phase(s) of the session are suitable for inclusion of the block;
a phase importance indicator, indicating the degree of importance for a phase of the session;
a duration indicator;
optionally a class specific parameter, indicating a parameter specific for a block class;
optionally an event identifier, indicating the event(s) in which the block can be used;
optionally an event merge identifier, indicating whether a block can or cannot be merged with other blocks;
optionally a theme identifier, indicating a theme if the block contains a language independent asset;
optionally language identifier, indicating a language if the block contains a language dependent asset.

Further provided is a system for generating at least a part of a therapeutic session comprising:

a) a block database module, comprising a plurality of blocks; and
b) a control module, configured for performing the method of generating at least a part of a script for a therapeutic session.

The block database module may comprise a first database comprising the plurality of blocks and a second database comprising a plurality of unique identity codes, each identity code associated with one or more of the blocks of the first database.

The control module may be configured for receiving a user input and modifying a stored session with respect to the received user input.

The user input may be one of the following:
one or more adjustable initialising parameters,
an indication of the dynamic session duration adjustment;
an indication of a dynamic event insertion.

The system may further comprise a media renderer configured for rendering a generated session.

Further provided is a computer program, or a computer program product directly loadable into the internal memory of a computer, or a computer program product stored on a computer readable medium, or a combination of such computer programs or computer program products, configured for performing the method according as described herein.

### DESCRIPTION OF THE FIGURES

The following description of the figures of the invention is only given by way of example and is not intended to limit the present explanation, its application or use. In the drawings, identical reference numerals refer to the same or similar parts and features.

FIG. 1 shows an exemplary visualisation of an exemplary therapeutic session comprising four phases.

FIG. 2 illustrates dynamic session duration adjustment for a session in progress.

FIG. 3 illustrates how the respiratory pattern of a subject can be regulated by means of breathing class blocks.

FIG. 4 illustrates dynamic event insertion for a session in progress.

FIG. 5 provides a more detailed example of a merge event insertion.

FIG. 6 shows a schematic illustration of a system for generating at least a part of a therapeutic session, the system (S) comprising a block database module (1100) and a control module (1000).

FIG. 7 shows a schematic illustration of a graphical user interface (100) for receiving input from the user (U).

In FIG. 8 a snapshot of an exemplary block's visual content is presented.

In FIG. 9 a second snapshot of an exemplary block's visual content is presented.

### DESCRIPTION OF THE INVENTION

As used below in this text, the singular forms “a”, “an”, “the” include both the singular and the plural, unless the context clearly indicates otherwise.

The terms “comprise”, “comprises” as used below are synonymous with “including”, “include” or “contain”, “contains” and are inclusive or open and do not exclude additional unmentioned parts, elements or method steps. Where this description refers to a product or process which “comprises” specific features, parts or steps, this refers to the possibility that other features, parts or steps may also be present, but may also refer to embodiments which only contain the listed features, parts or steps.

The enumeration of numeric values by means of ranges of figures comprises all values and fractions in these ranges, as well as the cited end points.

The term “approximately” as used when referring to a measurable value, such as a parameter, an amount, a time period, and the like, is intended to include variations of +/−10% or less, preferably +/−5% or less, more preferably +/−1% or less, and still more preferably +/−0.1% or less, of and from the specified value, in so far as the variations apply to the invention disclosed herein. It should be understood that the value to which the term “approximately” refers per se has also been disclosed.

All references cited in this description are hereby deemed to be incorporated in their entirety by way of reference.

Unless defined otherwise, all terms disclosed in the invention, including technical and scientific terms, have the meaning which a person skilled in the art usually gives them. For further guidance, definitions are included to further explain terms which are used in the description of the invention.

The present invention relates to a method of generating therapy or a therapeutic session (also called “session” herein) for inducing an altered state of consciousness (ASC) in a subject. An altered state of consciousness may be defined as a short-term change in the general configuration of one's individual experience, such that the rational functioning is clearly altered from one's normal state of consciousness. The altered state of consciousness is different from the state of being awake or asleep. The therapeutic session may include hypnosis therapy, which is a preferred method for inducing an altered state of consciousness in a subject. However, other psychological techniques may also achieve an altered state of consciousness. The hypnosis therapy may for example be medical (clinical) hypnosis therapy, or home hypnosis therapy, or hypnosis therapy provided in any care or wellness environment. While immersed in the altered state of consciousness the subject self-perception and the peripheral awareness are affected, changing the subject's experience of his/her sensation, perception, and thoughts, and making the subject prone to suggestive control of his/her physiological responses and movements.

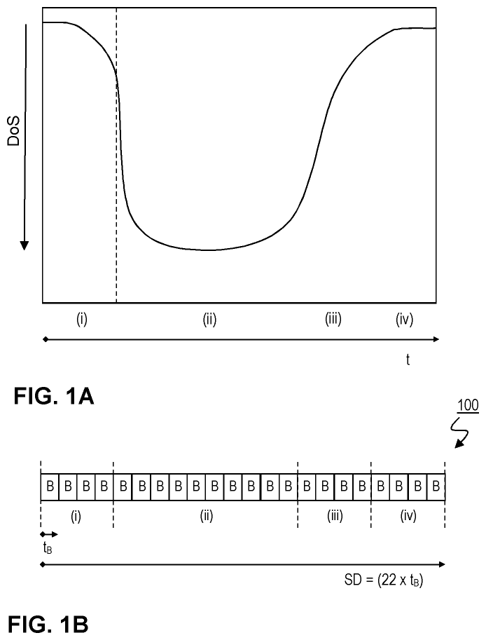

The therapy typically comprises four sequential phases as exemplified in FIG. 1A, namely:
(i) The induction phase, wherein the subject is prepared to be immersed into the altered state. In this phase the subject is typically provided with a feeling of comfort, safety and relaxation;
(ii) The deepening phase, wherein the subject is placed in an altered state of consciousness. This state is typically characterised by dissociation from reality and lack of or reduced movement unless expressly suggested in the therapy;
(iii) The transition phase, wherein the subject is exposed to suggestive information, which may aid in remembering or forgetting specific event of the therapy and/or to addressing one or more subject specific issues;
(iv) Re-alerting phase, wherein the subject is returned to a normal state of consciousness. In this phase the subject typically returns to a normal perception and the dissociative state ends.

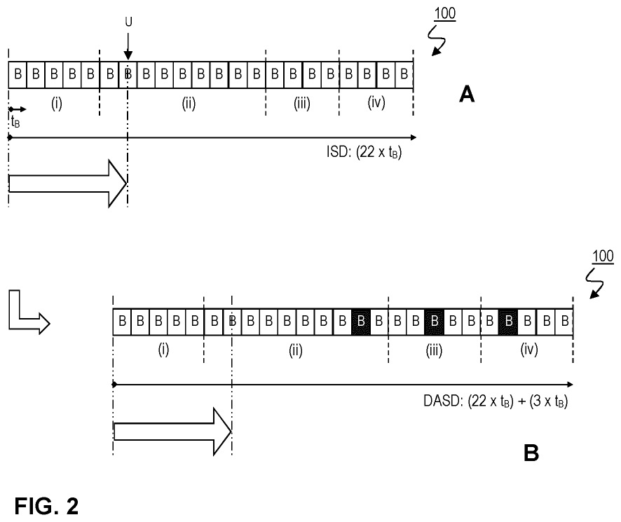

It is, however, noted that variations on the therapy may be available in the art; for instance adding new phases or modifying present phases to address case specific issues. The listed phases thus represent an overview of what may be considered to form the most rudimentary elements for performing the therapy. It will be therefore appreciated that the present methods and systems may be modified and the present disclosure is by no means limited to only the four listed phases. In the present disclosure, phases (i) to (iv) each comprise at least one “block” preferably more than one block, wherein a block is a short visual and/or audio segment; playing of the blocks in sequence generates the therapeutic session with an advantage that the session can be dynamically adjusted during the course of the session. An example of session comprising blocks is given in FIG. 2.

A used herein a “block” (also known as a building block, block package or block instance) refers to sensory information such as a visual and/or audio segment of predefined and fixed duration (e.g. 2-3 minutes). An executed block produces the visual segment and/or an audio track that can be experienced by the subject. The block is thus akin to a short scene in a motion picture and/or segment of an audio track. The block may contain a pre-defined visual and/or audio segment. The visual segment may be 2, or 3 three-dimensional, may be non-VR (virtual reality), or VR. A non-VR visual segment remains static when the subject's head moves relative to an external frame of reference of the subject. A VR visual segment is dynamically adjusted according to movement of the head of the subject relative to an external frame of reference when viewed on a VR headset. The audio segment may be mono or stereo or other (e.g. immersive sound, binaural, double induction, subliminal, surround or 3D audio effect). It may be non-VR or VR-responsive. A block is described here in terms of visual and/or audio content, though it is appreciated that the block contains data representing said visual and/or audio content to be rendered by a media render. Data regarding the block duration is associated with the block.

A block contains one or more assets. An asset may be visual or audio. An asset is an element (visual and/or audio) of a scene within the visual and/or audio segment of the block. A visual asset is a graphical element in a scene such as an ocean bed, sea, a plant or plants, or a sea creature—each may be a visual asset; typically it is moving. The data contained in the block may include data or a pointer to data defining the visual asset (e.g. exterior shape, colour etc.). An audio asset is a sound accompanying the scene, or independent to the scene, such as sound effects (e.g. from nature such as wind, sea, birds, waves), music or dialogue; each may be an audio asset. The data contained in the block may include data or a pointer to data defining the audio asset (e.g. midi or audio file).

The sensory information, such as a visual and/or audio asset, is configured to draw the subject's focus of attention through stimulation of the optical and/or auditory senses, respectively. The visual and audio assets may be synchronised to provide a simultaneous and potentially synergistic stimulation, or may be configured independently. Additionally or alternatively, the sensory information may also contain a somatosensory asset (e.g. warmth), a tactile asset (e.g. stimulate the mechanoreceptors by pressure or touch of skin), a kinaesthetic asset (e.g. stimulate the proprioceptors through movement of parts of the body) and/or an olfactory asset (e.g. stimulate the smell) configured to draw the subject's focus attention through stimulation of the proprioceptors and/or sense of smelling, respectively. It is understood that any embodiments described for visual and/or audio may also be apply for somatosensory and/or olfactory asset.

An asset may be a “principal asset” that is configured to be primary focus of attention by the subject. A visual asset many be a principal visual asset that is a moving artefact (e.g. whale, dolphin, or bird) or part thereof (e.g. tail or wing) that absorbs the attention of the subject; the artefact may for instance move at certain rate between different positions. The data contained in the block may include data or a pointer to data defining an exterior shape of a principal visual asset (e.g. whale, dolphin, bee or bird), a movement path of a part of the principal visual asset (e.g. whale tail, dolphin tail, bee stinger or bird wings), a movement trajectory of the principal visual asset (e.g. movement of whole whale or bee, dolphin or bird to left and right of a scene), an action performed by principal visual asset (e.g. landing of a dolphin or bird).

An audio asset may be a principal audio asset that provides the subject with auditory cues or script to focus on. The data contained in the block may include data or a pointer to data defining the principal audio asset (e.g. midi or audio file). The principal audio asset may include explanatory speech or suggestive sounds to make the subject more susceptible to certain aspects of the therapeutic session. The audio asset may be related to a visual asset, in particular an artefact of a visual asset. The data contained in the block may include data defining the sound a principal visual asset (e.g. whale or dolphin, sounds, buzzing of a bee or bird) or a movement or action performed by the principal visual asset (e.g. swimming of a whale or dolphin, flying of a bee or bird).

Aspects of the principal visual and/or audio asset are definable, such as the motion rate and extent of movement the principal visual asset (e.g. speed/extent of whale, dolphin, or bird movement) of a part of (e.g. speed/extent of tail or wing movement). The ability to adjust aspects of interventions may be implemented in the event class of block discussed later herein. The ability to adjust aspects of movement may be implemented in the breathing class of block discussed later herein.

An asset may be an “ancillary asset” that is configured to provide depth of immersion; it is typically not intended to be a primary focus of the attention of the subject. For instance, an ancillary visual asset may be a background element in a scene, such as landscape that moves as the subject proceeds forward in the session. Similarly, an ancillary audio asset may provide background music or relaxing sounds. The block may also contain only ancillary assets. The data contained in the block may include data or a pointer to data defining an exterior shape of the ancillary visual asset (e.g. seabed). The data contained in the block may include data or a pointer to data defining the ancillary audio (e.g. midi or audio file of background sounds).

A block may contain a mixture of principal assets and ancillary assets to compose the scene. A block may contain a mixture of principal visual and audio assets and of ancillary visual and audio assets to compose the scene.

The block may be assigned a block class depending on the intended function and effect of the block. Data regarding the block class is associated with the block.

Examples of block classes include:
A block assigned a “recess class” (recess block) provides (to the media renderer) a visual and/or audio segment configured to maintain the subject's immersion in the therapeutic session. The recess class block may comprise one or more assets that are a visual and/or audio element of the segment configured to maintain the subject's immersion in the therapeutic session; typically a recess class block will only contain ancillary assets allowing for rest or relaxation of the subject. Typically it does not include an additional effect in the subject.
A block assigned a “physiological class” (physiological block) provides (to the media renderer) a visual and/or audio segment configured to stimulate or regulate a physiological state in the subject. The physiological class block may comprise one or more assets that are a visual and/or audio element of the segment configured to induce or regulate the subject's physiological event and/or pattern; typically a physiological class block will comprise at least a principal asset to induce or regulate a physiological event and/or pattern. Examples include controlling the intensity of breathing, heartrate, pulse, movement of the body or specific parts of the body, opening or closing of the eyes.
A block assigned a “psychological class” (psychological block) provides (to the media renderer) a visual and/or audio segment configured to stimulate or regulate a psychological state in the subject. The psychological class block may comprise one or more assets that are a visual and/or audio element of the segment configured to induce or regulate the subject's psychological event and/or pattern; typically a psychological class block will comprise at least a principal asset to induce or regulate a psychological event and/or pattern. Examples include intensity of emotions, such as anxiety, fear, stress, and/or changes in behaviour.
A block assigned a “breathing class” (breathing block) provides (to the media renderer) a visual and/or audio segment configured to stimulate or regulate the respiratory pattern (e.g. inhalation and exhalation) in the subject. A breathing block is a specific type of physiological block. The breathing class block may comprise one or more assets that are a visual and/or audio element of the segment configured to regulate or synchronise the subject's respiratory pattern; typically a breathing class block will comprise at least a principal asset to synchronise the respiratory pattern. Examples of breathing class blocks are discussed further in the present description.
A block assigned an “event class” (event block) provides (to the media renderer) a visual and/or audio segment configured to guide the perception of the subject during the session e.g. in preparation for, anticipation of or facilitation of an episode external to or within the subject. An episode may be an external intervention to be performed near, on or to the subject, such as exposure to a loud noise or to a cold sensation. An episode may arise within the subject, such as labour and deliver. The event class block may comprise one or more assets that are a visual and/or audio element of the segment configured to guide the perception of the subject during the session; typically an event class block will contain at least a principal asset to draw the focus of attention away from the episode, or to integrate a specific moment of the episode into the therapeutic session through metaphors and/or suggestions. Examples of event class blocks are discussed further in the present description.
A block assigned an “outcome class” (outcome block) provides (to the media renderer) a visual and/or audio segment configured to stimulate or induce a specific (therapy) effect on the subject after the session e.g. to achieve an intended result from the session. For instance, block classes provided with suggestive information (e.g., changes in behaviour or emotion, remembering or forgetting specific events) for anxiety management (e.g. nausea, stress, fatigue, sleeping disorders), for pain management (e.g. chronic suffering), for sedation management (e.g. catalepsy, involuntary movement, suspension of reflexes). Examples of outcome class blocks are discussed further in the present description.
Other block classes may be created depending on the extent and purpose of the session. The skilled person understands that the present system and methods allows for integration of other block classes configured for other purposes.

Each block may be further assigned one or more block reference parameters (BRP) that provide operational and/or reference information regarding the block, for instance, block length, phase compatibility, combination compatibility, and the like. The one or more BRPs are associated with the block.

Examples of BRPs include:
Class indicator, indicating the class of the block, where a block class is indicative of a specific effect on a subject or lack thereof (e.g. breathing class block; event class block, recess class block); typically the class indicator may be assigned on the basis of a principal and/or ancillary asset contained in said block.
Phase identifier indicating which phase(s) (one or more of (i) to (iv)) of the session are suitable for inclusion of the block; the block may be assigned a single or multiple phase identifiers.
Importance identifier: indicating the degree of importance for a particular phase of the session; this identifier may be used to determine which blocks may be removed when shortening the duration of the session or used as padding to extend a session.
Duration indicator: indicating the duration (in sec or min) of the block.
Class specific parameter: blocks assigned a specific class may require one or more class specific parameters (e.g. the respiratory pattern for a breathing class block; the event type for an event class block);
Event identifier, indicating the event(s) in which the block can be used.
Event merge identifier, an indicator of which other blocks may be combined or merged with said block; for instance, a block containing recess audio content (soundtrack) which is language independent may be combined a block containing recess visual content. Typically the identifier will be determined on the presence or absence of a principal ancillary asset contained in said block.
Block frequency identifier, indicating the frequency a block may be included in a session; e.g. for single or multiple uses, e.g. looped.
Session frequency identifier, indicating the frequency a session may be performed on a subject; e.g. one-time procedure, repetitive acute situations, repetitive chronic situations.
Theme identifier: indicating a theme (e.g. ocean, sky, forest, etc.) if the block contains a language independent asset; this identifier may be attributed on the basis of a visual artefact (for a visual asset) or soundtrack (for an audio asset).
Language identifier, an indicator of the visual/audio content to be used which is language dependent; for instance a block containing an instruction track or presentable dialogue.
Culture identifier, an indicator of the visual/audio content to be used which is culture dependent; for instance a block containing an asset referencing a specific cultural subject.
Age identifier, an indicator of the visual/audio content to be used which is age dependent; for instance a block containing an asset configured for children or adults.
Cognition identifier, an indicator of the visual/audio content to be used which is cognition dependent, i.e. adjusted to the cognitive ability of the subject.

Each block containing one or more assets may be stored on a database, preferably, on a first database. Each block may be associated with a unique identity code. The unique identity code may be linked to the one or more block reference parameters (BRP).

The BRPs may be stored on the same (first) database as the block or may be stored on a second database; the unique identity code links the first and second databases. Preferably the block selecting comprises consultation of the second database to determine the blocks selected from the first database to generate at least part of the session.

It is to be understood that the above listed block features form an exemplary and non-exhaustive list, as more block features may be included or removed to adjust the efficiency of the selecting process, or to provide advanced features.

The method may allow the user or subject to interact with the progress bar of a GUI to select or highlight specific blocks. This may allow the user to see with one or more unique identity codes of the blocks. This could be particularly useful for customizing a stored script with a series of blocks.

As used herein the term “database” refers to a structured set of data typically stored on a storage media associated with a computing device; it may also perform various features and tasks to be performed, such as accessing, managing and updating of the stored data (blocks). The data in the database may be structured according to a database model; e.g. relational model, a hierarchical model, a network model, an analytical model or the like. The database may further be part of a network which includes at least one application (software) and/or at least one computing device (hardware). The network may provide access to the database from a plurality of computing devices and/or media renderers. A database may be a remote database, connected to the method by the Internet (open to possible tethered solution). A database may be held in a local storage medium or on the cloud.

As used herein the term “media renderer” refers to any device capable of rendering or playing the therapeutic session, in particular the blocks defined by the script that making up the session so it can be experienced by the subject. The playing may be interpreted broadly to include any type of content capable of stimulating the subject. Typically the ability to display video and/or emit audio content is a feature of any media renderer. Devices able to display video include a display panel (e.g. LCD, LED, OLED, other pixel-containing display panel), video projector (e.g. containing a DLP, LED or LED). Devices able to emit sound include a speaker, earpiece, or the like. The emitted sound may be mono, stereo, multidimensional etc. Preferably the media renderer is provided as a wearable device, e.g. headset. The media rendered may be provided as a virtual reality headset, as an augmented reality headset, or mixed reality headset. Preferably the media renderer comprises a display panel such as a screen, projector, or the like and a sound-emitting device, such one or more speakers, earpieces, or the like. Most preferably the media renderer is provided as a wearable device that provides virtual/augmented/mixed/etc. reality; which typically includes a display or projector, stereo sound (mono, stereo, multidimensional), and head motion tracking sensors (e.g. gyroscopes, accelerometers, structured light systems, etc.). Examples of suitable headsets are those supplied by Oculus (e.g. Oculus Rift, Oculus Go), LG electronics (e.g. LG 360 VR), HTC (e.g. HTC Vive), Samsung (e.g. Samsung Gear VR), Google (e.g. Google Cardboard). The media renderer may be expanded by also rendering somatosensory content, such as vibrations (e.g. vibration modules equipped in a seat), and/or olfactory content (e.g. releasing one or more fragrances into the nasal cavity). Optionally, the media renderer may be equipped with a computing unit for executing or playing data, or it may receive data from an external media server (i.e. streaming).

As used herein the term “subject” refers to a person experiencing the rendering or playing of the therapeutic session by the media renderer. The subject is the beneficiary of the therapeutic session. The “user” refers to a person or persons providing user input which may or may not be used to modify at least part of the therapeutic session. The user is an operator of the method. The user may be a care provider such as a physician or medical assistant. In some circumstances, the user may be the subject, for instance, where a therapy is self-administered at home. The rendered session draws the subject's focus of (visual and auditory) attention through stimulation of the (optical and auditory or other) senses. It does not require interaction with the subject, nor does it rely on any (sensory) input or (biometric) readings from the subject; it does not impose any cognitive burden on the subject. In other words, the information is presented to the subject for the purpose of producing in that subject a physiological reaction (e.g. focus and breathing) and/or a psychological reaction (e.g. behaviour and emotion), which can be measured by means of measurement systems (e.g. brain imagery, physical responses) during the therapeutic session.

As used herein the term “user interface” refers to the means by which the user and the computer system interact, which includes any potential input and display devices as well as the software for running said devices. Preferably the user interface is a graphical user interface (GUI), which provides the user with a visual way of interacting. For instance, the user interface may be the screen of a computer and a mouse/keyboard, or the touchpad of a tablet. Interaction with the user interface is limited to the user; it does not receive input from the subject. The user interface may comprise an input device of a computing unit, such as a touchscreen screen of a computer, tablet, smart phone or other smart device.

Described herein is a computer-implemented method for generating at least a part of a therapeutic session, the method comprising:
providing a first database comprising a plurality of blocks, each representing a visual and/or audio segment to be executed in its entirety,
generating a script defining at least a part of the therapeutic session, the script instructing sequential execution of at least 2 entire blocks selected from the first database;
storing the script for subsequent execution of the blocks whereby the visual and/or audio segments are presented to a subject during the therapeutic session.

The generated script contains instructions for sequential execution of at least 2 entire blocks selected from the first database.

A generated script typically comprises instructions for sequential execution of blocks corresponding to the aforementioned phases (i) to (iv), each phase comprising a plurality of blocks. Prior to a start of the session, the user may initialise the session by setting one or more adjustable initialising parameters that determine the blocks contained in the script that are to be used in each phase; the adjustable initialising parameters may include the total duration of the session and the respiratory pattern (described further below). Typically, the script may define a default session of for instance ˜25 minutes duration having a predefined sequence of blocks. The default session may be adjusted by the addition or subtraction of blocks according to the adjustable initialising parameters set by the user.

The method generates at least part of the script; it may generate a script for an entire phase making up part of the session; it may generate a script for at least part of a phase of the session; it may generate a sequence to be inserted or combined with at least a part of the session or phase; or it may generate the entire session. Alternatively, it may generate different parts of the session which can, post generation, be sequenced to obtain the entire session. Typically prior to playing, a script for the session will be generated in its entirety, whereas parts of the session script will be generated on user input (on demand) which may then be inserted into the session script or may replace at least a part of the session script. Scripts for sessions of predefined durations, such as the sessions most commonly used, may be pre-generated to conserve computing resources; these pre-generated scripts may then be similarly modified on user input.

The selection of blocks defined in the script may be dependent on the initialising parameters set by user and depend on the requirement and conditions of the session. As outlined above, a full session typically comprises at least four sequential phases. Each phase is characterized by different content intended to induce or maintain the subject's immersion/dissociation and/or control the subject's experience and physiological/behavioural/emotional/psychological response. Accordingly, the selecting of the blocks will be dependent on the phase selection. In case a full session is generated, the blocks will be sequenced in the script to match the order of the phases (i) to (iv). Further, the phases may each have predefined duration, which vary with respect to the total duration of the session. Accordingly, the selecting of the blocks will be dependent on the duration of the blocks, and of the phase or session. For example, a phase of a requested duration of 30 min may comprise 10 blocks of 3 min each to be executed sequentially, or it may comprise 30 blocks of 1 min each. The blocks may also be of different durations; for instance, comprising 10 blocks of 2 min each and 10 blocks of 1 min each. The duration of the blocks may vary depending on diverse characterizing parameters, such as the corresponding block class, identifiers, labels, and so on. Accordingly, the selecting of the blocks will also be dependent on said characterizing parameters (of the blocks).

After generation, the script may be stored for instance in temporary memory (cache), and/or on a storage medium (e.g. hard drive). The script may be stored in temporary memory (cache), and/or on a storage medium the computing unit generating the script, or temporary memory (cache), and/or on a storage medium of a computing unit executing the script (in case they are different). An example of a computing unit is a control module mentioned below. The script may alternatively be stored on another device in memory present in the media renderer. The script may alternatively be stored on a data centre to be accessed remotely (e.g. cloud computing).

The session may comprise at least four phases: (i) an induction phase, (ii) a deepening phase, (iii) a transition phase and (iv) a re-alerting phase, wherein each phase comprising at least partially different blocks, wherein the script for at least one phase is generated by the present method, preferably at least the deepening phase; preferably a script for all the phases is generated by the present method. The selection of phases to be (or not to be) generated may be an adjustable initialising parameter.

The script is typically code defining a plurality blocks for execution and an order of execution the blocks. Execution of the script causes blocks defined by the script to be executed. Execution of a block causes visual and/or audio segment defined by the script to be outputted for presentation to the subject via the media renderer.

The method may further comprise the steps of:
receiving a user input prior to generation of the script for the therapeutic session of a requested total duration of the session;
wherein the step of generating comprises selecting blocks from the first database such that a sum of the selected block durations corresponds with the requested total duration of the session.

Setting the total duration of a session may be a specific adjustable initialising parameter to be set by the user. A shorter session comprises fewer blocks than a default session. A shorter session may comprise fewer recess blocks, for instance, compared with a longer session. It is an aspect that the initialised total duration of session may result in change in duration of at least two phases compared with the default session, preferably all of the phases. Keeping the duration of the phases proportional may improve the subject's immersion and experience. The duration of the phases may be changed depending on a requested therapy outcome of the session, discussed further below.

The method generates the script for the session by selection of blocks of the appropriate duration. By reference to the importance identifier, blocks that are essential are retained, those that are non-essential can be removed or used as padding to extend a session. The class of block is also considered; blocks of the recess class are typically non-essential and can be removed or added with little or no impact.

The user input may be received through the aforementioned user interface. The method may accept values within a range; the values may be freely entered or selected from a discrete list of values. (e.g. 30 min, 35 min or 40 min). The discrete list of values may be displayed in the user interface by means of a dropdown menu. Upon receiving user input, the method generates the session of selected duration for storing, or it may request additional confirmation from the user.

The method may further comprise the steps of:
receiving a user input during execution of a current block of the therapeutic session of an adjusted total duration of the session;
modifying the script, by selecting one or more blocks for adding to or removing from the stored script, without interrupting complete execution of the current block such that a sum of the selected block durations extend or reduce the duration of the session according to the requested adjusted total duration of the session.

The modified script may be stored; in particular may replace the previously-stored script. The user may extend or shorten a session after a session has started, referred to as dynamic session duration adjustment. An example is shown in FIG. 2 herein. Dynamic session duration adjustment allows the current block to run to completion, while adding one or more subsequent blocks to the session or removing one or more subsequent blocks in the session that have not yet been executed. This avoids interruption of an existing block at the moment a request by the user for a session duration adjustment has been is received (which could potentially break a subject's immersion). Where a session is to be extended, one or more subsequent blocks may be inserted into the session between existing blocks or after an existing block. Where a session is to be shortened, one or more subsequent blocks may be removed from the session.

The method may select the block of appropriate duration for adding or removing by reference to the importance identifier; blocks that are essential are retained, those that are non-essential can be removed or used as padding to extend a session. The class of block is also considered by reference to the class indicator.

The session may comprise at least the four phases: (i) an induction phase, (ii) a deepening phase, (iii) a transition phase and (iv) a re-alerting phase, wherein the (ii) a deepening phase is the longest phase. The deepening phase (ii) may be about as long as the sum of the other (i, iii, iv) phases; preferably at least as long as the sum of the other (i, iii, iv) phases, for example twice as long. The deepening phase (ii) is the phase wherein most of the subject stimulation (e.g. respiratory control, events) may take place; it is therefore typically the longest phase. For example, in a session of about ˜45 min total duration the deepening phase may have a ˜30 min duration. Preferably, the dynamic session duration adjustment may result in change in duration of at least two, preferably all of the current and remaining phases. The change in duration may be proportional. By keeping the duration of each phase proportional, the subject's immersion may be improved. For instance, a dynamic session duration adjustment receiving deepening phase (ii) may result in a change in duration of the current session (deepening phase (ii)), and in each of the subsequent sessions-transition phase (iii) and re-alerting phase (iv).

The method may comprise providing an output for a graphical user interface indicating progress of the session and total duration of the session. The timing progress may be presented visually via a progress bar, optionally with a progress timer and/or a countdown timer until the end of the session. Additionally, the total session duration and/or progress within the session responsive to a request for session duration adjustment can be (dynamically) displayed on the graphical user interface. The (dynamic) visual representation not only provides a more intuitive understanding to the user, but also provides a reduction of resource consumption at the computing unit.

The first database may further comprise one or more “outcome class” blocks (outcome class block or outcome block herein). The sensory information such as the visual and/or audio segment of an outcome class block induces a specific (therapy) effect on the subject during and/or after the therapeutic session. The effect may be a clinical effect; i.e. relating to clinical hypnosis. Selection of outcome class block may allow for easily adjusting the session to achieve an intended result on the subject without risking disruption of the altered state of consciousness. As a result, outcome class blocks may contribute to customizing the method to a subject's specific needs and improving the success rate of the session. Also, outcome class blocks may allow for keeping the session simple and intuitive for a user; without needlessly complicating the execution of a session, the user can focus on the task to be performed during the session.

The session outcome may be configured prior to generation of the script. This may typically include the selection of primary therapy outcomes; i.e. pre-planned with regard to the subject's need of the therapeutic session, which were identified prior to the execution of the session.

The method may further comprise the steps of:
receiving a user input prior to generation of the script of a requested outcome of the session;
wherein the step of generating comprises selecting from the first database at least 1 outcome class block comprising an asset that is sensory information such as a visual and/or audio element of the segment configured to achieve a specific effect on the subject during and/or after the session.

The therapy outcome may be configured during execution of the script (i.e. during the session). This may typically include the selection of secondary therapy outcomes; i.e. responsive to specific subject needs that might be identified during the therapeutic session.

The method may further comprise:
receiving a user input during execution of the script of a requested outcome of the session;
selecting from the first database at least 1 outcome block responsive to the user input;
inserting into the stored script an instruction for execution of the selected outcome block without interruption of the execution of the current block.

The inserting of the at least 1 outcome class block into the stored session may comprise:
inserting the selected outcome block between two existing blocks in the script to be executed after the current block; and/or
merging the selected outcome block with one or more existing blocks in the script to be executed during or after the current block.

Examples of block insertion or merging are presented further in the present description.

The user input may be provided through a selection phase, which permits a user to configure the therapeutic session to the subject's needs. The selection of a therapy outcome may determine the selection of blocks, in which case the blocks indicated with the requested class indicator are selected instead of the class blocks of standard therapeutic session. Additionally or alternatively, the selection of a requested therapy outcome may allow for the introduction of additional blocks into the script; the scripts may have an enhancing effect on specific parts of the session.

Examples of requestable therapy outcomes include:
discomfort management, configuring the degree of comfort provided by the present session. In particular, reducing or preferably removing the sensation of unease and discomfort caused by specific disorders such as anxiety, nausea, fatigue, which might occur prior to, during or after the procedure. The selected blocks provide an anxiolytic effect on the subject.
sensitivity management, configuring the degree of desensitization provided by the present session. In particular, reducing the reaction to physical or sensory events, which may happen during or after the present procedure, e.g. sensations to which the subject may be exposed. The selected blocks provide a desensitizing effect on the subject.
dissociation management, configuring the degree of dissociation provided by the present session. In particular, controlling the immersion of a subject in the present session, e.g. disconnection from normal thoughts, feelings, memories, or surroundings. The selected blocks provide a dissociative effect on the subject.
pain management, configuring the degree of analgesia provided by the present session. This may include reducing the intensity of pain or distress in the subject, and/or the reaction to a pain stimulus by the subject. The selected blocks provide an analgesic effect on the subject.
sedation management, configuring the degree of sedation provided by the present session, In particular, by limiting the partial or full movement range of the subject. This may include a limitation on the movement of a specific body part, such as the head or arms, up to catalepsy of the full body. The selected blocks provide a sedating effect on the subject

Selection of specific therapy outcomes may have an impact on the selection of other block classes. Certain outcome blocks may not be combinable with other block classes.

The first database may further comprise one or more “breathing class” blocks (breathing class block or breathing block herein). At least one of the blocks in the first database may be classified as a breathing class block. A block that is assigned a “breathing class” (breathing class block or breathing block herein) contains an asset configured to induce a respiratory pattern in the subject; the asset for inducing the breathing is referred to as the principal asset of the breathing class block. The principal asset may be visual (e.g. a moving graphical element) and/or audio (e.g. repetitive sounds) to which the breathing of the subject is synchronisable. An example of a principal visual asset is whale, dolphin, or bird where a part of the asset such as tail or wing has a regular movement that can be adjusted according to the desired inhalation and exhalation duration. The visual and/or audio segment of a breathing class block induces a regulated respiratory pattern.

The respiratory pattern refers to a duration of inhalation and exhalation. The breath class block may induce a breathing pattern of defined inhalation and exhalation duration. The respiratory pattern may be constant for a duration of the block. Setting the respiratory pattern for at least a part of a session may be another specific adjustable initialising parameter.

Thus a breathing class block can be used to adjust to the standard breathing pattern (of the subject), such as increasing or decreasing the breathing pace. A standard breathing pattern of a subject at rest is typically composed of approximately 3.5″ (seconds) of inhalation followed by an approximately 3.5″ of exhalation (3.5/3.5). However, it is noted that subject's suffering from a breathing disorder (e.g. anxiety or reduced lung capacity) may have a divergent breathing pattern, such as 2.0/2.0, 2.5/2.5 or lower. A paediatric subject might have smaller lung capacity and thus also require shorter breathing patterns.

As indicated above, adjustments to the standard breathing pattern may affect the heart rate variability (HRV), which in turn may affect the autonomous nervous system. Accordingly, the breath class block produces a physiological effect which does not depend on psychological or cognitive factors. The physiological reaction (e.g. respiratory pattern, HRV) may be measured by means of physical parameters. Assets and by extension breathing blocks containing those assets may be regarded as functional data configured to control, inter alia, the respiratory pattern of the subject during the therapeutic session. Preferably the respiratory patterns are regulated during the (ii) transition and/or (iii) deepening phase. The breathing class blocks may be limited to (ii) transition and/or (iii) deepening phase.

The breathing class block regulates the respiratory rate and/or the inhalation-to-exhalation ratio. The respiratory rate or alternatively breathing pace is defined as the number of breaths taken per minute. The inhalation-to-exhalation ratio is defined as the ratio between the duration of inhalation and the duration of the exhalation in seconds. For example, a subject with a 3.5″/3.5″ breathing pattern (i.e. 3.5 seconds inhalation/3.5 seconds exhalation) (ratio of 1:1) will have a respiratory rate of 8.5 breaths per minute. Changing the breathing pattern by decreasing the respiratory rate from for instance 3.5/3.5 to 5.0/5.0 may increase the HRV and activate the parasympathetic nervous system.

The method may generate a script containing at least two breathing class blocks to be executed sequentially; wherein a first breathing class block has a respiratory pattern corresponding with a standard respiratory pattern, and a second breathing class block has a respiratory pattern corresponding with an adjusted respiratory pattern. For example, for a subject having a respiratory rate of 3.5/3.5 the first breathing block may have a corresponding respiratory rate of 3.5/3.5, to which the subject's breathing may synchronise, and the second breathing block may have a respiratory rate of 5.0/5.0, to decrease the subject's breathing rate accordingly. By providing a sequence of breathing class blocks, a first breathing class block being synchronising with the subject's standard respiratory pattern and a second breathing class block a longer inhalation and/or exhalation duration, the subject may more effectively enter the deepening phase.

The respiratory pattern may be regulated by providing a plurality of breathing blocks to be executed sequentially, each block defining a constant respiratory pattern for a duration of the block, and is evolved with respect to an adjacent and/or proximate block. The evolution may be a longer or shorter inhalation and/or exhalation duration compared with a previous adjacent block. Preferably a subsequent block may have a longer inhalation and/or exhalation duration compared with a previous adjacent block. A subsequent block may, however, have a shorter inhalation and/or exhalation duration compared with a previous adjacent block. In a sequence of breathing blocks, the respiratory pattern may be incremental or decrease between adjacent blocks.

For example, a first breathing block may have a respiratory rate of 3.5/3.5, a second breathing block may have an incrementally increased respiratory rate of 4.0/4.0, the third may breathing block may have an incrementally increased respiratory rate of 4.5/4.5, and the fourth breathing block may have an incrementally increased respiratory rate of 5.0/5.0. This sequence may regulate the subject's breathing rate to decrease incrementally. By regulating the respiratory pattern in incremental steps, the subject may more effectively enter the deepening phase.

The incremental regulation may be percentage (%) based; the respiratory pattern of the sequential breathing blocks may increase or decrease based on a % of the adjacent and/or proximate blocks. A subsequent block may have a percentage difference in inhalation and/or exhalation duration compared with a previous block. A subsequent block may have a percentage increase in inhalation and/or exhalation duration compared with a previous block. A subsequent block may have a percentage decrease in inhalation and/or exhalation duration compared with a previous block. Preferably, the percentage is 10 to 50%; more preferably 20 to 40%; most preferably 25 to 35%; for example 33%. A % based regulation might be implemented when the subject suffers from a (temporary) breathing disorder without increasing the total duration of the session.

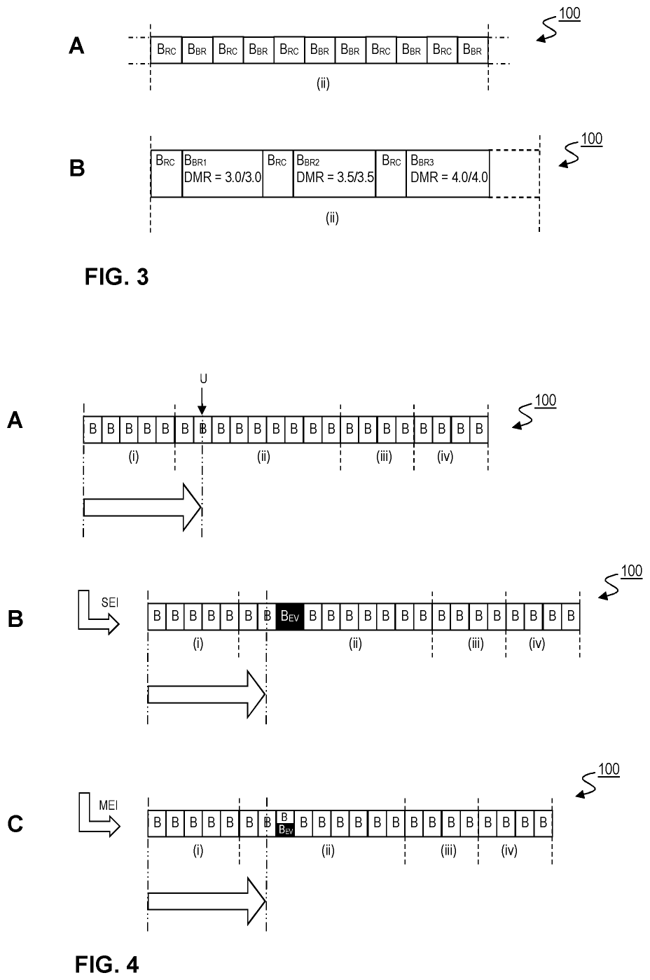

An asset, in particular a principal asset, or a part of the principal asset of the breathing class block may have a definable motion rate (DMR). The DMR refers to a regular movement of the asset or principal asset, or a part thereof. For example, a tail part of a principal asset that is a dolphin may sweep at a definable rate. It may be defined by a change in position of the asset or part thereof between a first region and a second region within a set time interval. The DMR may be constant for the duration of the block. The respiratory pattern of the subject synchronises to the DMR. The DMR is preferably constant for a duration of a first breathing block, and also be constant but different for a duration of a subsequent breathing block. The DMR may be smaller or greater in a subsequent breathing block, preferably slower. In an example, the principal asset of a breathing block may be a dolphin having a sweeping tail; the sweeping rate of the dolphin's tail is set with a DMR of 3.5″, which results in a complete tail sweep from side to side within a 3.5″ time interval. By rendering in a first breathing block the sweeping motion of the dolphin's tail at a 3.5″ interval the subject may synchronise his/her (standard) respiratory rate to the DMR. Next, in the subsequent breathing block the tail sweeping interval may be decreased to 5.0″ (5.0 sec interval); which will stimulate the subject to decrease his/her respiratory rate to match the slower DMR. As a result thereof, the subject's respiratory rate will have decreased substantially by synchronising with the DMR of the visual asset. In a further example the DMR may be increased incrementally (4.0″ to 4.5″ to 5.0″). An example of a progressively incremental DMR is shown in FIG. 3.

The starting DMR and final DMR may have default values (e.g. 3.5/3.5 starting DMR, 5/5 final DMR), or may be adjustable by the user (user input). An adjustable starting DMR might be implemented when the subject suffers from a (temporary) breathing distress, for instance exhibiting a faster respiratory rate. By providing a capability of an adjustable starting DMR, the DMR can be synchronised to the breathing pattern of the subject, from which he can be brought more effectively into a slower breathing pattern.

The asset of the breathing class block may include audio element (audio asset, or a principal audio asset). The audio asset or a principal audio asset may repeat at a set time interval to which the respiratory pattern of the subject synchronises. The audio asset may be a sound clip with cues repeating at moments of inhalation and exhalation. Continuing the example above, the audio cues may be gushes of water or breath sounds preferably synchronised with the DMR of the visual asset (i.e. the sweeping motion of the dolphin's tail). The audio principal asset might be stand alone, independent of any principal visual asset.

The method may comprise providing an output to a graphical user interface indicating the respiratory pattern (duration of inhalation and/or exhalation) and/or progress of the current breathing block and optionally the respiratory patterns and running times of the following or remaining breathing blocks. The timing may be presented via a progress bar, or a countdown timer, or other visual indicators. By providing a visual indication of the progress, the user might better prepare for the execution of the task associated with the respiratory pattern. Additionally, the GUI may provide the user with bar visualising the inhalation and exhalation. For instance, if the respiratory pattern is intended to induce a (ii) deepening phase in the subject, the user may organise tools to initiate any procedures as soon as the deepening phase is induced. This overview allows for the session to be executed more efficiently, thereby reducing the occurrence of potential stress on both user and subject's end.

The first database may further comprise one or more “event class” blocks (event class block or event block herein). The sensory information such as a visual and/or audio segment of an event class block prepares the subject for an episode external to or within the subject. It may guide the attention of the subject away from the episode or integrate a specific moment of the episode into the dissociative experience through metaphors and/or suggestions. Preferably, presentation of a visual and/or audio segment of an event class block to the subject may be synchronised with the occurrence of the event. The event class block may allow for performing the episode without disrupting the altered state of consciousness of the subject. The event class block may thus contribute to improving the mental and physical health of a subject during otherwise stressful or painful events.

The method may further comprise the steps:
receiving a user input during execution of the script of the therapeutic session of a requested event;
selecting from the first database at least 1 event class block;
inserting into the stored script an instruction for execution of the corresponding event block without interruption of the execution of the current block.

The event block may contain one or more assets or principal assets configured to prepare the subject for the episode and/or guide or distract the subject during occurrence the episode. The event block may contain a plurality of visual assets and/or a plurality of principal visual assets. The event block may contain a plurality of audio assets and/or a plurality of principal audio assets which distract or overload the senses of the subject, thereby altering the self-perception and peripheral awareness.

Accordingly, the information presented (as rendered session) produces in the mind of the subject an effect which does not systematically depend on psychological or cognitive factors but on a (stimulatory or inhibitory) physiological reaction. Accordingly, stimulatory or inhibitory assets and by extension event blocks containing those assets should be regarded as functional data configured to control, inter alia, the physiological reaction (of the subject) during the therapeutic session. Engaging the subject with sensory information of an event class block may improve the efficiency of the episode. In particular, the user may increase focus on performing the episode without worrying about subject's response or interaction. Also, the safety of the subject may be improved, by increasing the performance and success rate of the user.

Examples of events may include:
Pain: the event block induces an analgesic effect (in the subject) to minimise reaction to pain, such as from needles or incisions.
Cleaning: the event block induces an effect to minimise reaction to cleaning of the subject, such as cleaning of the subject's skin.
Probing: the event block induces an effect to minimise reaction to insertion of a probe, or similar devices and cables.
Temperature: the event block induces an effect to minimise reaction to warm or cold sensation.
Smell: the event block induces an effect to minimise reaction to specific smells, such as burning skin.
Deeper respiration: the event block induces an effect to inhale or exhale deeply.
Pausing respiration: the event block induces an effect to momentarily stop breathing; for instance, during acquisition of a radiological image
Labour: the event block prepares the subject for experiences of labour and delivery.

The one or more assets or principal assets of the event class block may be adjusted to the nature of the specific event. For example, the audio and/or visual assets presented for a pain event may be different than those for a cleaning event. As indicated earlier in this description, specific input parameters, like selection of a specific therapy outcome, may impact the event class block selection. For example, if the user indicated that a subject may experience from high levels of anxiety, the event class block length may be increased. For example, if the user indicated that a higher level of dissociation or pain management is required, the event class block intensity (i.e. amount of assets used) may be increased.

The event block may be inserted to the script upon user request after the session has been initialised and while the session is still running, also referred to as dynamic event insertion. An example of dynamic event insertion is shown in FIG. 4. The event block is inserted into the script after the currently executed block; the currently executed block is allowed run to completion. This avoids interruption of an existing block at the moment of request by the user for insertion of an event block (which could disrupt the immersion). The system may also determine an optimal time for insertion of the event class block; for instance, following a specific narrative or metaphors of the therapeutic session. Insertion of the event class block may then be delayed until the blocks of the current narrative are finished.

Dynamic event insertion during execution of the script, i.e. after the script has been initialised and while the session is still running, forms an interactive therapeutic session between a user (e.g. a practitioner) and a subject. This may free up the user's time to prepare the necessary resources or to respond to unforeseen complications without risking disrupting the immersion of the subject.

Insertion might in some cases, for specific events, superimpose on an already running or future block, if the superimposed event class block is referenced as having the ability to superimpose. It is a further aspect allows for removal of an added event block from the script, for instance events that are deemed as no longer necessary or were input by accident. If removal is requested, the method may comprise selecting one or more new blocks to replace the removed event blocks, or it may revert the previously selected blocks.

Upon receiving a user input, the selected event block will be inserted into the script. The updated script is stored. The user typically does not know in advance when the event will take place; the event block may be inserted at any moment within a certain window of time, for instance, during phase (ii), upon receipt of a request. The user may request no time delay for the event; the event is to be performed as soon as possible. Execution of the current block is not interrupted and the requested event block is inserted as soon as possible after execution of the current block. Alternatively, the user may also set a time delay for the event; the time delay may for example be required for preparation of the event. Execution of the current and future blocks is not interrupted and the requested event block is inserted as soon as possible, but not before the set time delay has passed. Additionally, certain events may require a (mental) preparation period before the event or (mental) relaxation period after the event to maintain full immersion. This is important whenever a series of events is requested by a user. In this case, the event may further dictate a period of relaxation to follow prior to insertion of the following event.

The selected event block may be inserted in the script as a block between two existing blocks so splitting them apart, referred to as split event insertion. An example of split event insertion is shown in FIG. 4B. When split inserted, the event block will essentially become a new block in the session extending the total duration. In case the extended duration is undesired by the user, the split inserted event may trigger the removal of one or more subsequent blocks of similar duration. One or more recess blocks may for instance be removed from the session in such case, which has little adverse stimulatory effects. Information on resulting extended session and/or an option to reduce the resulting total session length may be conveyed to the user.

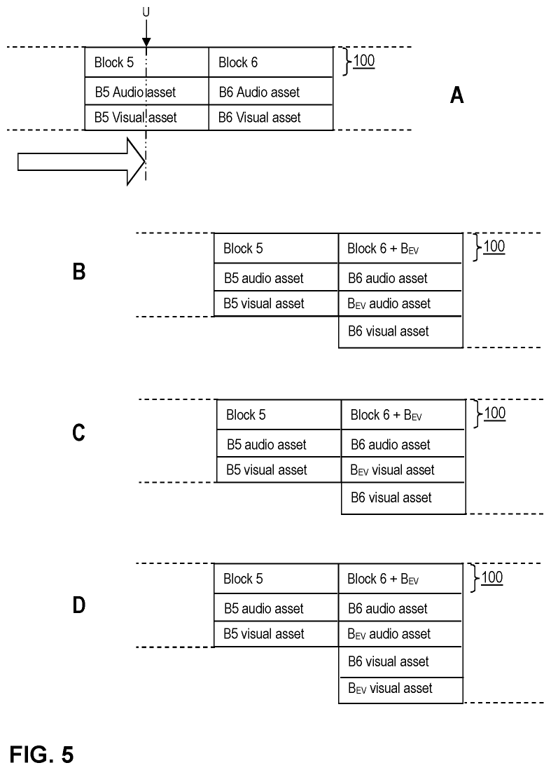

The selected event block may be inserted into the script as block superimposed over an existing block, referred to as merge event insertion. An example of merge event insertion is shown in FIGS. 4C and 5. This way the asset(s) of the event block may be merged together with the asset(s) of the existing block. In certain cases the merging may be preferred over split insertion as it does not result in any time extension of the total duration of the session. However, not all blocks are compatible for merging. For instance, if an existing block contains a principal asset that could interfere or inhibit the principal asset of the event block, the merging cannot be performed (without disrupting the subject's immersion). The compatibility of blocks may be indicated using block identifiers, in particular using the combinatory identifier. Preferably the merging is performed over one or more recess blocks, which contain ancillary assets and thus have little adverse stimulatory effects. For example, an event block may be inserted as block containing a visual asset superimposed over an existing block (visual merging event block); or an event block containing an audio asset may be superimposed over an existing block (audio merging event block); or an event block containing visual and audio assets may be superimposed over an existing block (visual and audio merging event block). It is understood that embodiments related to event class block insertion or merging may also relate to insertion or merging of other class type blocks.

The method may comprise (the step of) informing the user of the time until execution of the requested event block. Notifying the user of the time until execution of the requested event allows for better synchronising of the event with the principal assets of the event class block. This may enhance the feeling of immersion and also reduce the risk of breaking the altered state of consciousness of the subject. The time until execution may be presented through various means, such as visual or audio means. An example of a visual means is a countdown timer presented on a display as discussed below. An example of an audio means is a series of audio cues generated by speaker (not hearable by the subject).

The method may comprise (the step of) providing an output for a graphical user interface indicating the insertion of a (selected) event block into the progress of the session. The user may be able to monitor the remaining time before insertion of the event via the GUI. For instance, the GUI may provide the user with a (visual) overview of the session progress and indicators at which time the event block will be inserted. The progress may be presented via a progress bar, optionally with a countdown timer, or other timing means. By providing a visual overview of the session progress towards an event, the user might better prepare for the execution of the task associated with the event. For instance, if the event is intended to minimise reaction to insertion of a probe into the subject, the user might already prepare the tools required for said insertion, such as providing lubricant onto the probe.

It is understood that the method and in particular the visualisation of the event block timing does not necessarily assist the user in performing the task; it may guide the user in better executing the timing of the task. The visualisation of the event occurrence and real-time adjustment thereof (allowing execution of the event block at the earliest opportunity) allow the user to see and optionally interact with the internal state of the underlying technical system. The real-time adjustment allows for the event to be executed faster, more efficiently (i.e. using less processing resources) and with greater control (over duration and timing). This enables the user to properly operate the underlying complexities of the technical system dynamically without risking malfunctions or undesired physical responses of the subject (e.g. by missing or extending portions of event class blocks).

During or prior to receiving the user input, the GUI may present an overview of selectable events which are associated with one or more event class blocks. The overview (of selectable events) may be text based or visually represented (e.g. icons, symbols, tokens, models, etc.; optionally colour coded), or a combination of both. The visual representation provides a more intuitive understanding to the user by displaying images relevant for the associated input. It may also result in a reduction of resource consumption at the computing unit and/or media renderer by resolving conflicting events automatically (e.g. incompatible series of events or timings). The events may be grouped by event types; selection of an event type may open a secondary list with subclasses of events or event variations. This allows for a cleaner and more intuitive representation of selectable events.

During or after receiving the user input, the GUI may present a visual aid for the user specific for the selected event(s). The visual aid may be text-based or visually represented (e.g. icons, symbols, tokens, models, etc.; optionally colour coded), or a combination of both. The visual aid may be designed to more easily convey information to the user, for instance by visualisation in an enlarged portion of the display device and maintaining an overview of a zone of interest which is larger than the initial GUI display area. The visual aid may assist the user in performing a technical task (associated with the event) by means of a continued and/or guided human-machine interaction. Preferably the visual aid is connected to the session duration, starting before or together with the rendering of the selected event block.

In a preferred embodiment of the present invention, the method may comprise: providing a first database comprising a plurality of blocks, each representing a visual and/or audio segment to be executed in its entirety, the database comprising at least one or more recess class blocks comprising an asset configured to maintain a subject's immersion during the therapeutic session, and one or more event class blocks comprising an asset configured to induce a specific (therapy) effect on the subject during the therapeutic session; generating a script defining at least a part of the therapeutic session, the script instructing sequential execution of at least 2 entire blocks selected from the first database; storing the script for subsequent execution of the blocks whereby the visual and/or audio segments are presented to the subject during the therapeutic session;

wherein during execution of the stored script, preferably during the therapeutic session, the method further comprises: receiving a user input of a requested event; selecting from the first database at least 1 event block responsive to the user input; inserting into the stored script an instruction for execution of the selected event block without interruption of the execution of the current block; optionally, informing the user of the time until execution of the requested event block.

It is an aspect of the present invention to provide a system for generating at least a part of a therapeutic session, the system comprising
a) a block database module, comprising a plurality of blocks; and
b) a control module, configured for performing the method of generating a script defining at least a part of a therapeutic session.

Preferably the block database module comprises a first database comprising the plurality of blocks and a second database comprising a plurality of unique identity codes, each identity code associated with one or more of the blocks of the first database.

The control module is configured for generating the script by selecting from the first database on the block database module at least 2 blocks to be executed sequentially; and storing the script. The control module may be configured to generate a default script comprising instructions for execution of a plurality of sequentially arranged blocks corresponding to at least one phase of a session, preferably to phases (i) to (iv) of the session.

The control module may further read the stored script, access the first database according to the script and output audio and/or visual segments for rendering by media renderer.

The control module may be configured for receiving a user input and modifying the stored script with respect to the received user input. The user input may for instance be:
One or more adjustable initialising parameters (e.g. total duration of the session, respiratory pattern),
An indication of the dynamic session duration adjustment (i.e. extending or shortening the duration of the session)
An indication of a dynamic event insertion (e.g. one insertable event, optionally a series of insertable events).

The control module may receive several inputs at once. The user inputs may be received sequentially or simultaneously, preferably sequentially. In case a series of insertable events is requested, each event may be inserted according to the input sequence. The control unit may provide feedback to the user of the selected event via the GUI; this may allow the user to cancel a requested event. In case a series of insertable events is requested, the sequencing may also be adjusted by the user. This allows the user to interact with the internal system state of the underlying machine.

The control module may generate a graphical user interface (GUI) which provides the user with a visual overview of selected session parameters, and preferably provides feedback on user input. The GUI may visualise the progress of the session (e.g. progress bar), and optionally one or more visual indicators or timers. For instance, the GUI may provide the user with an overview of the current respiratory pattern and the timing of when the respiratory pattern will change. For instance, the GUI may provide the user with an overview of the requested events and indicators at which time the event block will be inserted

The control module may generate a history log of the session. This log may comprise a (chronological) overview of requested user input prior or during the session; the log may also indicate the timing of request and timing of execution. Additionally, the log may comprise a series of statistics of the session, such as the total running duration of the session or a specific phase. The log may be stored on a storable device for consultation at a later time. The log may be transferred (e.g. via the Internet) to a session log database, containing data for a plurality of sessions

The system may further comprise a media renderer configured rendering the session, presenting it to the subject. The (visual and/or audio) assets of the session are rendered on a media renderer, such as a virtual reality headset. Signals corresponding to the (visual and/or audio) assets of the session that are received by the media renderer may be generated by a computing device associated with the control module (e.g. computer) or with the media renderer itself (e.g. smartphone). The control module typically regulates the block execution to allow the media renderer to render the current block. The control module may pre-regulate the following block to allow the media renderer to provide seamless transition between the current block and the following resource. The media renderer may be a headset configured for virtual, augmented, mixed reality or the like. The media renderer may be a smartphone inserted into a wearable device. The media renderer is connected or connectable to the control module; the connection may be wired or may be wireless (e.g. Bluetooth connection or a WiFi connection, or other wireless protocol).

It is an aspect of the invention to provide a computer program, or a computer program product directly loadable into the internal memory of a computer, or a computer program product stored on a computer readable medium, or a combination of such computer programs or computer program products, configured for performing the method as described herein.

It is an aspect of the invention to provide a kit for performing the method as described herein; the kit comprising
a media renderer
a computer program configured for performing the method as described herein.

The kit may comprise a computing device preconfigured with the computer program for performing the method as described herein.

### EXAMPLES

### Example 1: Therapeutic Session

FIG. 1A shows an exemplary visualisation of a typical hypnotic therapeutic session comprising four phases performed in sequence wherein the x-axis represents time (t) passed and the y-axis the depth of altered state of consciousness of the subject, also referred to as Depth of State (DoS). The four sequential phases in this example refer to (i) the induction phase, (ii) the deepening phase, (iii) the transition phase, and the (iv) re-alerting phase. While immersed in the therapeutic sessions the subject's self-perception and the peripheral awareness are affected, the immersion depth increasing during the (i) phase and reaching a maximum during the (ii) phase. The subject becomes prone to suggestive control during the (ii) phase, such as using breathing class blocks to control the subject's respiratory pattern or event class blocks to control the subject's physiological reactions or movement. Afterwards the subject is returned to a normal state of a consciousness by passing the (iii) and (iv) phases. Further details of the phases may be found discussed in the description above.

FIG. 1B further shows how a script (100) for a therapeutic session (comprising the four phases as illustrated in FIG. 1A) can be formed from a plurality of blocks (B), each block defining a (visual and/or audio) asset of fixed duration to be executed in its entirety. In this particular example each block has the same fixed duration (t_{B}). Each phase (i)-(iv) contains a series of blocks. For instance, phase (ii) contains ten blocks. Accordingly, the total duration of the therapeutic session (session duration, SD) corresponds to the sum of the duration of each block which in this particular example results in 22 blocks multiplied with t_{B}.

### Example 2: Dynamic Session Duration Adjustment

In FIG. 2 an example is provided of how the total duration of a session may be dynamically adjusted during the session by amendments to the script (100). Dynamic session duration adjustment allows the current block to run to completion, while adding or removing one or more subsequent blocks to the script that have not yet been executed. This avoids interruption of block being executed at the moment a request by the user for a session duration adjustment has been is received (which could potentially break a subject's immersion). The user may extend or shorten a session after a session has started; in this particular example the user decides to extend the session with a time corresponding to 3 blocks during the phases (ii), (iii), and (iv).

The Panel A shows a stored script (100) (in line with example 1) with an initial session duration (ISD) corresponding with 22 blocks to be executed in sequence. The user provides input (U) during the execution and rendering of the seventh block. The current session progress is represented with the arrow. After receiving user input the system determines the optimal position for insertion of additional blocks between or after existing blocks in the script. Panel B illustrates that 1 block (shown with a black background) has been added to places in the script (100) corresponding to each phase that is running or is yet to run; in particular phases (ii)-(iv). By keeping the duration of each phase proportional, the subject's immersion and experience may be improved. The dynamically adjusted session duration (DASD) corresponds with the 22 initial blocks extended with 3 additional blocks. For the sake of simplicity all blocks are shown to have the same running duration (t_{B}). This may of course be varied depending on more specific user input to include values that are not integers of the standard blocks.

### Example 3: Breathing Class Blocks

In FIG. 3 an example of a script (100) containing breathing class blocks to regulate the respiratory pattern (e.g. respiratory rate and/or the inhalation-to-exhalation ratio) of a subject in a therapeutic session. The subject's respiratory pattern is regulated in the (ii) phase through execution of six breathing blocks (B_{BR}) partially dispersed between recess blocks (B_{RC}) in the script (100) shown in Panel A. The executed breathing blocks provide (to the renderer) stimulations to regulate the respiratory pattern in the subject. The recess blocks provide stimulations intended to maintain the subject's immersion. By alternating the breathing and recess blocks the subject's respiratory pattern may be controlled with lesser risk of fatiguing/overventilating the subject.

Panel B shows a script (100) specifying three of the breathing blocks, wherein content of each breathing block is shown in greater detail, B_{BR1}, B_{BR2}, B_{BR3}. The respiratory pattern (of the subject) may be controlled by means of a graphical element (visual asset) having a definable motion rate (DMR) to which the respiratory pattern of the subject synchronises; wherein the DMR is defined by a change in position of the graphical element between a first region and a second region at a set time interval. In this particular example, the DMR of breathing blocks 1 to 3 (B_{BR1} to B_{BR3}) gradually increases (from 3.5″/3.5″ to 4.0″/4.0″) leading to a gradually slower breathing pattern (increased inhalation/exhalation duration).

### Example 4: Event Class Blocks

In FIG. 4 an example is provided of how an event class block (B_{EV}) may be inserted in the script (100) of a therapeutic session. In Panel A, the user (U) (e.g. physician, subject) requests an event after execution of the script (100) has started; in this particular example the user decides to request an event session during the (ii) phase. The event class block is inserted to the script upon user request after the session has been initialised and while the script is still running, also referred to as dynamic event insertion. Dynamic event insertion allows insertion of an event block into the script during execution of a current block, and allows the current block to run to completion. In the present example two methods are illustrated for dynamic event insertion.

In Panel B, the event block (B_{EV}) is inserted as a block containing visual and optionally audio content between two existing blocks. This first method is referred to as split event insertion (SEI) which splits apart two previously adjacent blocks. A request to insert a split event block is received while executing a current block (upper panel) in phase (ii). Execution of the current block is not interrupted and the requested event block (B_{EV}) is inserted (split insertion) between two existing blocks as close as possible to the current block (first lower panel). Split insertion of an event block will result in a longer session run time unless another (optional) block is removed to compensate and keep the total time of the session as defined by the user.

Alternatively, the event block might in some cases, for specific events, superimpose on an already executing or future block as shown in Panel C. The (visual and/or audio) asset of the (selected) event block will in this case be merged with the asset of an existing block. This second method is referred to as merge event insertion (MEI). In certain cases the merging may be preferred over split insertion as it does not result in any time extension of the total duration of the session. However, not all blocks are compatible for merging. For instance, if the block contains a principal asset that could interfere or inhibit the principal asset of the event block, the merging cannot be performed (without disrupting the subject's immersion).

FIG. 5 provides a more detailed example of a merge event insertion (MEI) into a script (100), and corresponding content of the blocks. In the present example (Panel A) the user (U) requests, during the execution of Block 5, a specific event to be executed as soon as possible. Execution of the current block (Block 5) is not interrupted and the system determines that the event block is suitable for merging with Block 6. In a first possibility (Panel B) the requested event block (B_{EV}) contains only audio content, and the modified script (100) instructs execution resulting in the audio content of the Block 6 being merged with audio content of B_{EV}. In a second possibility (Panel C) the requested event block (B_{EV}) contains only visual content, and the modified script (100) instructs execution resulting in the visual content of Block 6 being merged with visual content of B_{EV}. In a third possibility (Panel D) the requested event block (B_{EV}) contains both audio and visual content; the modified script (100) instructs execution resulting in merged audio and visual content of Block 6 and B_{EV}.

An example of an event block insertion to induce an analgesic effect (in the subject) during executing of the therapeutic session is presented. The subject is maintained in an altered state of consciousness using a series of recess class blocks. At a certain point during the therapeutic session, the user determines that an event requires the use of a needle, to which the subject is expected to have an adverse reaction. The user of the sessions requests a ‘needles event’ via the GUI of the system. The system determines that the time of insertion of the corresponding event block without disrupting the immersion of the subject, e.g. without interrupting the audio or visual content of the recess class blocks. The system calculates the time until exaction of the requested event block and informs the user thereof via a countdown timer presented on a display of the system. The user may use the remaining time to prepare the necessary resources. Upon finishing of the countdown timer, the user may synchronise the use of the needle with the audio and/or visual content of the event class block. It is understood that similar examples may be provided for other the use of other event class blocks, such as those corresponding to the use of a cleaning item, a probing means, changes in temperature, smells, ambient sound, and so on.

### Example 5: System

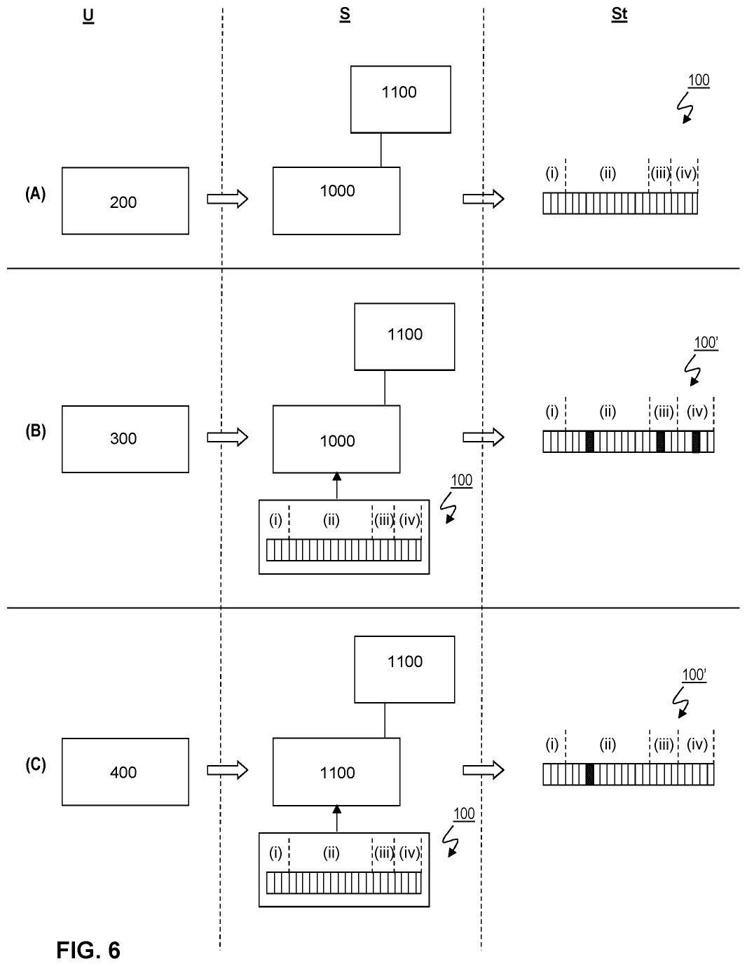

FIG. 6 shows a schematic illustration of a system for generating a script (100) for at least a part of a therapeutic session, the system (S) comprising a block database module (1100) and a control module (1000). FIG. 6 further shows how the system (S) may generate and store (St) a therapeutic session script (100, 100′) responsive to user input (U).

In the first, upper panel (A) the initialisation of the session is shown. The system receives a series of adjustable initialising parameters from the user (U), such as session duration and DMR. The inputted values are received by the control module (1000) which selects a plurality of blocks from the block database module (1100) to generate a session comprising at least four phases and matching with the received parameters.

In the second, middle panel (B) the session is running and a dynamic duration adjustment is shown. The system receives a request from the user (U) to increase the duration of the session. The control module (1000) checks the status of the session in progress and determines suitable points for inserting additional blocks selected from the block database module (1100). The modified script (100′) is stored (St) which replaces the script (100) in progress.

In the third, bottom panel (C) the session is running and a dynamic event insertion is shown. The system receives a request from the user (U) to insert an event in the stored script (100). The control module (1000) (error on the FIG. 6C? 1100 mentioned in the control module) checks the status of the session in progress and determines a suitable point for inserting the event block. The requested suitable event block is selected from the block database module (1100). The modified script (100′) is stored which replaces the script (100) being executed.

### Example 6: GUI

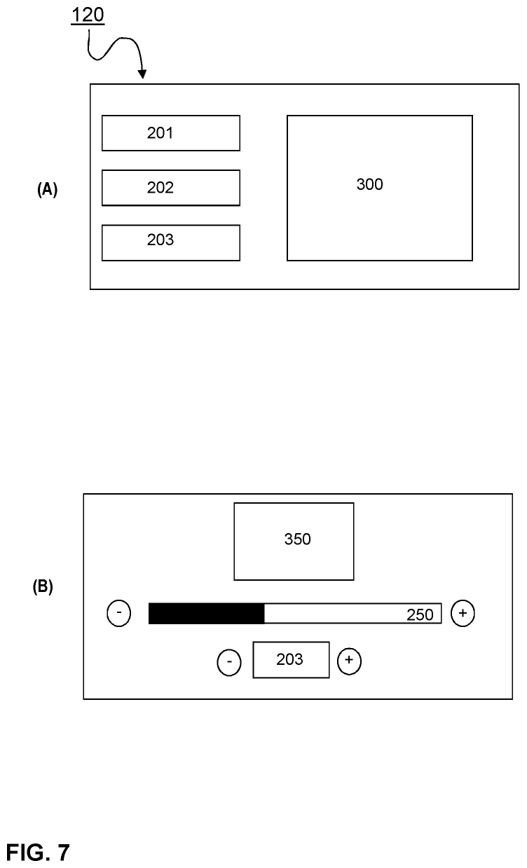

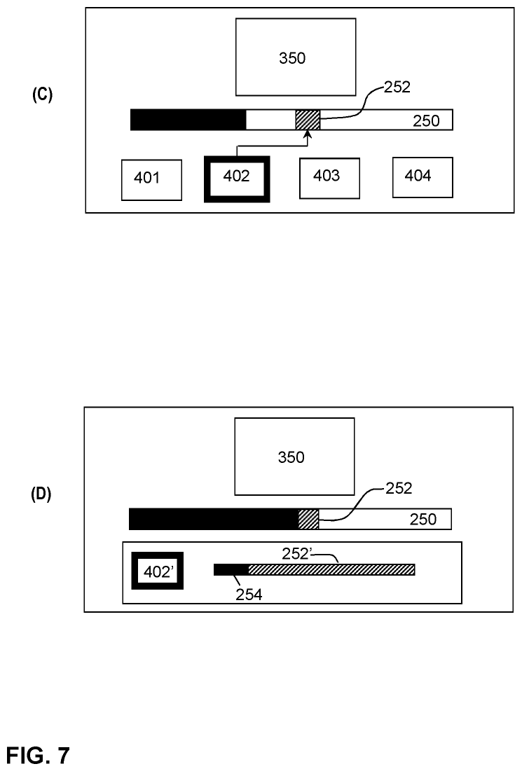

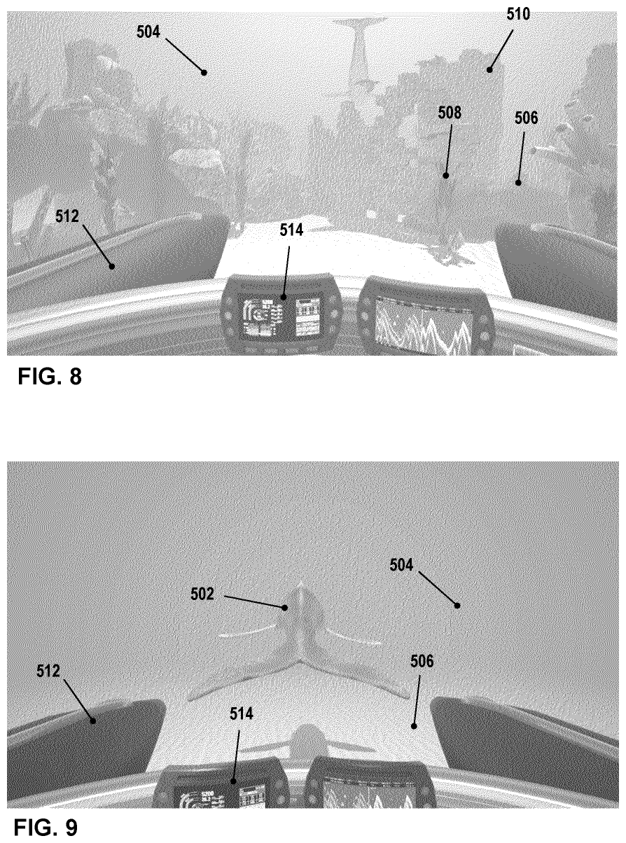

FIG. 7 shows a schematic illustration of a graphical user interface (120) for receiving input from the user (U).

In the first panel (A) the initialisation of the session is shown. The GUI allows the user to input a series of adjustable initialising parameters, such as language (201), session duration (202) and PMR (203). The GUI may also present a preview of the session (300) and optionally select a theme of the session.

In the second panel (B) the session is in progress and the rendered media is shown on a display (350). Below the display a progress bar (250) is shown which indicates the lapsed section of the session in black. The progress may optionally be supplemented with a countdown timer, or other timing means. The user may extend or shorten a session by interacting with the GUI, for instance by pressing the (+) and (−) buttons, respectively. Below the progress bar (250) the PMR indicator shows the current motion rate regulating the subject's respiratory pattern. The user may increase or lower the PMR by interacting with the GUI, for instance by pressing the (+) and (−) buttons, respectively.

In the third panel (C) the session is also in progress and the rendered media is similarly shown on a display (350). Below the progress bar (250) a series of interactive buttons (401, 402, 403, 404) is displayed, each corresponding with a specific event such as pain, cleaning, probing, temperature, smell, and so on. The buttons (of selectable events) may be text based or visually represented (e.g. icons, symbols, tokens, models, etc.; optionally colour coded). The user may select one event, such as (402), which will cause the system to insert the event block associated with said event in the session. The insertion may be visually indicated on the progress bar (250) as indicated with the crossed portion (252).

In the fourth panel (D), the event is being executed and displayed on the display (350). The progress bar (250) indicates the time point in the session, and that the event (252) potion is being executed. Below, an expanded event (252′) potion is displayed together with a label (402′) of the name of the event, and the progress bar indicates elapsed time (254) of the event.

### Example 7: Assets

In FIG. 8 a snapshot of an exemplary block's visual content is presented. The block comprises a plurality of ancillary assets intended to support the subject's immersion. In this particular example the ancillary assets include the water (504), the seabed (506), the plants (508), ruins (510), and the submarine housing (512) and dashboard (514). By moving the seabed the impression is created that the submarine is moving forward in the video. None of the ancillary assets are intended to catch the attention of the subject, but provide immersion was well as a calm relaxing atmosphere instead. The shown snapshot is therefore part of a recess block. Where the block is rendered by a virtual reality headset, the subject is able to survey by movement of the head the entire scene as if projected around a sphere. The block may also be provided with audio assets, such as ocean sounds or calming music and/or voice.

In FIG. 9 a snapshot of an exemplary block's visual content is presented. The block comprises a principal asset which is an artefact of a whale (502) located in a central position intended to maintain the attention of the subject. The block further comprises several ancillary assets intended to support the subject's immersion. The ancillary assets include the water (504), the seabed (506) and the submarine housing (512) and dashboard (514). By moving the seabed the impression is created that the whale is swimming forward in the video while being followed by the subject in the submarine.

Continuing this particular example, the position of the may whale's tail may start sweeping from a first region (top) to a second region (bottom) at a predefined speed or time interval; defined as the predefined motion rate (PMR). This can stimulate the subject to synchronise his/her respiratory pattern with the PMR of the sweeping tail. Additionally, the block may be provided with principal audio assets synchronised with the PMR of the sweeping tail; for example repeating audio cues. If the block comprises such principal assets configured to regulate the respiratory pattern of the subject, the block will be classified as a breathing class block.

The assets shown in FIGS. 8 and 9 may be varied thematically while keeping the core concept of the session intact. For instance, instead of the above described aquatically themed session an alternative location (e.g. forest, sky, and so) may be selected for the assets. Naturally calming themes tend to be better suited for stimulating and maintaining the subject's immersions. For example, in an air themed session the ancillary assets may the sky and clouds, whereas the principal assets may be flying birds. The flapping of the bird's wings may be defined as the PMR. For example, in a forest themed session the ancillary assets may be (still) trees and plants, whereas the principal asset may be a butterfly. The flapping of the butterfly's wings may be defined as the PMR.

1. A computer-implemented method for generating at least a part of a therapeutic session, the method comprising:
providing a first database comprising a plurality of blocks, each block representing a sensory information, preferably a visual and/or audio segment, to be executed in its entirety;
generating a script defining at least a part of the therapeutic session, the script instructing sequential execution of at least 2 entire blocks selected from the first database;
storing the script for subsequent execution of the blocks whereby the sensory information, preferably visual and/or audio segments, are presented to a subject during the therapeutic session;
wherein during execution of the stored script the method comprises:
receiving a user input of a requested event;
selecting from the first database at least 1 event class block comprising an asset that is sensory information, preferably as a visual and/or audio element of the segment, configured to guide the perception of the subject;
inserting into the stored script an instruction for execution of the selected event block without interruption of the execution of the current block;
preferably informing the user of the remaining time until execution of the selected event block.

2. The method according to claim 1, wherein the inserting of the at least 1 event class block into the stored session comprises:
inserting the selected event block between two existing blocks in the script to be executed after the current block; and/or
merging the selected event block with one or more existing blocks in the script to be executed during or after the current block.

3. The method according to claim 1, wherein the method comprises providing an output for a GUI indicating the insertion of the selected event block into the progress of the session, and preferably providing a visual indicator indicating the remaining time until execution of the selected event block, preferably via a countdown timer.

4. The method according to claim 1, further comprising:
receiving a user input prior to generation of the script of a requested outcome of the session;
wherein the step of generating comprises selecting from the first database at least 1 outcome class block comprising an asset that is sensory information, preferably a visual and/or audio element of the segment, configured to induce a specific therapy effect on the subject.

5. The method according to claim 1, further comprising:
receiving a user input during execution of the script of a requested outcome of the session;
selecting from the first database at least 1 outcome block comprising an asset that is sensory information such as a visual and/or audio element of the segment configured to induce a therapy specific effect on the subject;
inserting into the stored script an instruction for execution of the selected outcome block without interruption of the execution of the current block.

6. The method according to claim 1, further comprising:
receiving a user input prior to generation of the script of a requested total duration of the session;
wherein the step of generating comprises selecting blocks from the first database such that a sum of the selected block durations corresponds with the requested total duration of the stored session.

7. The method according to claim 1, further comprising:
receiving a user input during execution of a current block of an adjusted total duration of the session;
selecting one or more blocks for adding to or removing from the stored script without interruption of the execution of the current block such that a sum of the selected block durations extend or reduce the duration of the session according to the requested adjusted total duration of the stored session.

8. The method according to claim 1, wherein the method comprises providing an output for a graphical user interface (GUI) indicating progress of the session and total duration of the session.

9. The method according to claim 1, wherein at least one of the blocks in the first database is classified as a breathing class block comprising an asset that is a visual and/or audio element of the segment configured to induce a defined respiratory pattern in the subject, preferably a defined respiratory rate and/or the inhalation-to-exhalation ratio.

10. The method according to claim 9, wherein the method selects at least two breathing class blocks to be played sequentially; wherein a first breathing class block has a respiratory pattern corresponding with a standard respiratory pattern of the subject, a the second breathing class block has a respiratory pattern corresponding with an adjusted respiratory pattern.

11. The method according to claim 10, wherein the method selects a sequence of at least two breathing class blocks to be played sequentially; wherein each breathing class block in the sequence defines a constant respiratory pattern and is evolved with respect to an adjacent breathing class block.

12. The method according to claim 11, wherein the evolution is an incremental change optionally that is 10 to 50%; more preferably 20 to 40%; most preferably 25 to 35%; for example 33% of the inhalation and/or exhalation duration.

13. The method according to claim 9, wherein the asset of the breathing class block is at least one visual or audio asset, the asset or part thereof having a definable motion rate, DMR, to which the respiratory pattern of the subject synchronises, wherein the DMR is defined by a change in position of the graphical element between a first region and a second region within a set time interval.

14. The method according to claim 9, wherein the asset of the breathing class block is at least one audio asset repeating at a set time interval to which the respiratory pattern of the subject synchronises.

15. The method according to claim 9, wherein the method comprises providing an output for a GUI indicating the respiratory pattern and progress of the current breathing block, and optionally the respiratory patterns and running times of a following block.

16. The method according to claim 1, wherein the therapeutic session comprises at least two phases of: (i) an induction phase, (ii) a deepening phase, (iii) a transition phase and (iv) a re-alerting phase, wherein each phase comprising at least partially different blocks; and wherein a script defining at least the deepening phase, and one other phase is generated.

17. The method according to claim 1, wherein each block is assigned a unique identity code in the first database, and a second database contains a list of unique identity codes of the first database, each unique identity code associated with a block reference parameter providing reference information about the block, wherein the selecting comprises consultation of the second database to determine the blocks selected from the first database to generate the therapeutic session.

18. The method according to claim 17, wherein a block reference parameter associated with the unique identity code is one or more of the following:
a class indicator, indicating the class of the block, where a block class is indicative of an effect on a subject or lack thereof;
a phase identifier, indicating which phase(s) of the session are suitable for inclusion of the block;
a phase importance indicator, indicating the degree of importance for a phase of the session;
a duration indicator;
a block frequency identifier,
optionally a class specific parameter, indicating a parameter specific for a block class;
optionally an event identifier, indicating the event(s) in which the block can be used;
optionally a session frequency identifier;
optionally an event merge identifier, indicating whether a block can or cannot be merged with other blocks;
optionally a theme identifier, indicating a theme if the block contains a language independent asset;
optionally language identifier, indicating a language if the block contains a language dependent asset;
optionally culture identifier, indicating a culture if the block contains a culture dependent asset;
optionally age identifier, indicating an age group if the block contains a age dependent asset;
optionally cognition identifier, indicating a cognition group if the block contains a cognition dependent asset.

19. A system for generating at least a part of a therapeutic session comprising
a) a block database module, comprising a plurality of blocks; and
b) a control module, configured for performing the method of generating at least a part of a script for a therapeutic session.

20. The system according to claim 19, wherein the block database module comprises a first database comprising the plurality of blocks and a second database comprising a plurality of unique identity codes, each identity code associated with one or more of the blocks of the first database.

21. The system according to claim 19, wherein the control module is configured for receiving a user input and modifying a stored session with respect to the received user input.

22. The system according to claim 21, wherein the user input is one of the following:
one or more adjustable initialising parameters,
an indication of the dynamic session duration adjustment;
an indication of a dynamic event insertion.

23. The system according to claim 19, wherein the system further comprises a media renderer configured for rendering a generated session.

24. The system according to claim 19, configured for performing a method for generating at least a part of a therapeutic session, the method comprising:
providing a first database comprising a plurality of blocks, each block representing a sensory information, preferably a visual and/or audio segment, to be executed in its entirety;
generating a script defining at least a part of the therapeutic session, the script instructing sequential execution of at least 2 entire blocks selected from the first database;
storing the script for subsequent execution of the blocks whereby the sensory information, preferably visual and/or audio segments, are presented to a subject during the therapeutic session;
wherein during execution of the stored script the method comprises:
receiving a user input of a requested event;
selecting from the first database at least 1 event class block comprising an asset that is sensory information, preferably as a visual and/or audio element of the segment, configured to guide the perception of the subject;
inserting into the stored script an instruction for execution of the selected event block without interruption of the execution of the current block;
preferably informing the user of the remaining time until execution of the selected event block.

25. A computer program, or a computer program product directly loadable into the internal memory of a computer, or a computer program product stored on a computer readable medium, or a combination of such computer programs or computer program products, configured for performing the method according to claim 1.
